(19) United States
(12) Patent Application Publication
(10) Pub. No.: US 2002/0091139 A1
(43) Pub. Date: Jul. 11, 2002

(54) Treatment and delivery of hydroxychloroquine

(75) Inventor: B. Lauren Charous, Fox Point, WI (US)

Correspondence Address: SCULLY, SCOTT, MURPHY & PRESSER, 400 Garden City Plaza, Garden City, NY, 11530, US

(73) Assignee: DAMON TODD HOLDINGS, L.L.C., FOX POINT, WI

(21) Appl. No.: 10/055,148

(22) Filed: Jan. 22, 2002

Related U.S. Application Data
(63) Continuation of application No. 08/953,087, filed on Oct. 17, 1997.

(51) Int. Cl. A61K 31/47

(52) U.S. Cl. 514/313

(57) ABSTRACT

The treatment of various disease states with hydroxychloroquine.

### BACKGROUND OF INVENTION

Hydroxychloroquine (HCQ) and other members of this anti-malarial class of drugs have been administered per ora and been found useful as therapeutic systemic anti-inflammatory agents for a variety of serious inflammatory and auto-immune diseases such as rheumatoid arthritis and lupus erythematosus for much of the last half century.

HCQ has a remarkably wide range of actions including antagonist activity to histamine and antigen-induced bronchospasm and reduction of antibody responses, particularly those involving production of allergic antibody. Other actions relevant to the anti-asthmatic anti-allergic actions contained in this application include alterations in cell-mediated immunity, alterations in mediator effects and pathways, including evidence for inhibition of the arachidonic pathway cascade which results in the production of potent spasmogenic and inflammatory mediators such as leukotrienes, some novel and potentially important antiviral actions as well as other significant miscellaneous anti-inflammatory effects. For many years it has been known that anti-malarials antagonize histamine-induced bronchoconstriction in an animal model since as early as 1973 (Ayitey-Smith E, Boye G L. Effect of Chloroquine on Histamine-induced Bronchial Asthma in the Guinea Pig. J Pharm Pharmac 1974;26:208-09) and also reproducibly decrease antigen-induced bronchoconstriction in guinea pig trachea as demonstrated by Andersson. See, Andersson P. Effects of inhibitors of anaphylactic medicators in two models of bronchial anaphylaxis in anesthetized guinea pigs. British Journal of Pharmacology 1982;77:301-307.

Recent research has attributed increased importance of allergic antibody (IgE) in the pathogenesis of asthma. See, Goldstein R, Paul W, Metcalfe D, et al. NIH conference: Asthma. Ann Nit Med 1994;121(9):698-708. Immuno-modulation of humoral responses by HCQ is well described and is thought to be due to inhibition of antigen presenting cell (APC) antigen presentation in a variety of ways. See, Blum J, Cresswell P. Role for intracellular proteases in the processing and transport of class II HLA antigens. Proc Natl Sci USA 1988;85:3975-79. See, Grey H, Chesnut R. Antigen processing and presentation to T cells. Immunol Today 1985;6:101. See, Guidos C, Wong M, Lee K. A comparison of the stimulatory activities of lymphoid dendritic cells and macrophages in T proliferative responses to various antigens. J Immunol 1984;133:1179-85. See, Nowell J, Quaranta V. Chloroquine affects biosynthesis of Ia molecules by inhibiting dissociation of invariant (γ) chains from α-β dimers in B cells. J Exp Med 1985;162:1371. Fox described decreased IgA and IgG levels among patients with Sjogren's syndrome treated with HCQ. See, Fox R I, Chan E, Benton L, et al. Treatment of Primary Sjogren's Syndrome with Hydroxychloroquine. A J Med 1988;85(suppl 4A):62-7. Marsh and co-workers reported on the abrogation of IgE production by a human T-cell clone by pretreatment of APC with chloroquine. See, Shau-Ku H, Shinomiya N, Marsh D. Molecular and functional studies of human T-cell responses to a short ragweed pollen allergen AMB a V. JACI 1991;87(1:2):258A. The recent observation by Charous and co-workers that HCQ modulates IgE production appears relevant in this regard. See, Sherman B, Charous B. Short-term effects of hydroxychloroquine (HCQ) on IgE levels and obstructive airways disease. Journal of Allergy and Clinical Immunology 1992;89(1:2):286 (abstract).

Asthmatic inflammation is thought to be due to both the effects of a variety of potent cytokines and interleukins, arachidonic acid metabolites and the direct effects of lysosomal and cellular toxins released by neutrophils, basophils and eosinophils. Evidence accrues that anti-malarial agents may engender potent suppression of all of these avenues of inflammatory response.

HCQ is thought to inhibit T-cell participation in the immune response both through the inhibition of APC antigen presentation (see references above) coupled with direct alteration of surface markers on lymphocytes [and platelets], (Fox R, Kang H. Mechanism of action of antimalarial drugs: inhibition of antigen processing and presentation. Lupus 1993;2(1):S9-12.) (Nordhagen R, Flaathen S. Chloroquine removal of HLA antigens from platelets for the platelet immunofluorescence test. Vox Sang 1985;48:156-59.) (Nagarkatti P, Nagarkatti M, Jain V. In vivo and in vitro action of chloroquine on surface markers of human peripheral lymphocytes. Clin Exp Immunol 1980;41:166-72.) findings which appear relevant in light of recent reports documenting a central role of T-cells in the genesis of asthma, sarcoidosis and atopic dermatitis. See, Barnes P. Anti-inflammatory therapy for asthma. Annual Rev Med 1993;44:229-42. See, Garssen J, Nijkamp F, Van der Vliet H, et al. T-Cell-mediated induction of airway hyperreactivity in mice. Am Rev Respir Dis 1991;144:931-38. See, Leung D. Atopic dermatitis: the skin as a window into the pathogenesis of chronic allergic diseases. J Allergy Clin Immunol 1995;96(3):302-18. See, Robinson D, Hamid Q, Tsicopoulos A, et al. Predominant T(H2)-like bronchoalveolar T-lymphocyte population in atopic asthma. NEJM 1992;326(5):298-304. See, Walker C, Kaegi M, Braun P, et al. Activated T cells and eosinophilia in bronchoalveolar lavages from subjects with asthma correlated with disease severity. JACI 1991;88(6):935-42.

In ex vivo studies, HCQ inhibits both leukotriene and histamine release from human lung. See, Kench J G, Seale J P, Temple D M, et al. The Effects of Non-steroidal Inhibitors of Phospholipase A2 on Leukotriene and Histamine Release from Human and Guinea Pig Lung. Prostaglandins 1985;30(2):199-207. This finding is not unexpected, given the fact that the anti-malarials have found common usage as phospholipase A2 inhibitors in a wide variety of tissues, (Matsuzawa Y, Hostetler K Y. Inhibition of Lysosomal Phospholipase A and Phospholipase C by Chloroquine and 4,4′-Bis (diethyaminoethoxy) α,β-diethyldiphenylethane. J Bio Chem 1980;255:5190-94.) (Kubo M, Hostetler K. Mechanism of cationic amphiphilic drug inhibition of purified lysosomal phospholipase A1. Biochem 1985:6515-6520.) although it appears unlikely that this action is seen at the concentrations reached in vivo. The fact that anti-malarials appear to inhibit the formation of the leukotrienes suggests they may act by reducing late-phase reactions caused by antigen.

A possible role for cytokines in the genesis of the asthmatic reaction has recently been postulated. These cytokines, such as IL-1, IL-6 and tumor necrosis factor, are elaborated by alveolar macrophages, bronchial epithelial cells and human lung fibroblasts. See, Takizawa H, Ohtoshi T, Kikutani T, et al. Histamine activates bronchial epithilial cells to release inflammatory cytokines in vitro. Nit Arch Allergy Immunol 1995;108(3):260-67. See, Zitnik R, Whiting N, Elias J. Gudocorticoid inhibition of interleukin-1-induced interleukin-6 production by human lung fibroblasts: evidence for transcriptional and post-transcriptional regulatory mechanisms. Am J Respir Cell Mol Biol 1994;10(6):643-50. HCQ and analogues inhibit the release IL-1 and IL-6 from T-cells and monocytes and tumor necrosis factor from human macrophages in vitro; (Sperber K, Quraishi H, Kalb T, et al. Selective regulation of cytokine secretion by hydroxychloroquine: inhibition of IL-1 and IL-6 in monocytes and T cells. J Rheumatol 1992;20(5):803-08.) Sperber, 1991 #89; Gosset, 1991 #91; Picot, 1991 #129.

The direct cytotoxicity of superoxides elaborated by a variety of cells, but especially neutrophils and eosinophils, is also substantially inhibited by the actions of anti-malarials. Recent reports have indicated that free-radical activity is heightened in asthma and may substantially contribute to airway inflammation. See, Owen S, Pearson D, Suarez-Mendez V, et al. Evidence of free-radical activity in asthma. NEJM 1991;325(8):586-87. HCQ and other anti-malarials markedly reduce neutrophil superoxide production and release as well as in peripheral blood monocytes. See, Hurst N P, French J K, Betts W H, et al. Evidence that Antimalarial Drugs Act as ‘Phospholipid Analogue’ Inhibitors of Key Pathways of Phospholipid Metabolism in Leucocytes (abst). B Journ Rheum 1986;25:127. See, Hurst N, French J, Bell A, et al. Differential effects of mepacrine, chloroquine and hydroxychloroquine on superoxide anion generation, phospholipid methylation and arachidonic acid release by human blood monocytes. Biochem Pharmacol 1986;35:3083-89. See, Hurst N P, French J K, Gorjatschko L, et al. Studies on the Mechanism of Inhibition of Chemotactic Tripeptide Stimulated Human Neutrophil Polymorphonuclear Leucocyte Superoxide Production by Chloroquine and Hydroxychloroquine. Ann Rheum Dis 1987;46:750-756. See, Miyachi Y, Yoshioka A, Imamura S, et al. Anti-oxidant Action of Antimalarials. Ann Rheum Dis 1986;45:244-48. Other miscellaneous anti-inflammatory actions by which HCQ could exert an important effect on bronchial and allergic inflammation include stabilization of lysosomal membranes. See, Weissman G. Labilization and Stabilization of Lysosomes. Fed. Proc. 1984;23:1038. Although no reports delineate the effect of HCQ on eosinophils, the breadth of its actions—ranging from membrane stabilization to decreases in lysosomal enzyme activity—(Gonzalez-Noriega A, Grubb J, Talkad V, et al. Chloroquine inhibits lysosomal enzyme pinocytosis and enhances lysosomal enzyme secretion by impairing receptor recycling. J Cell Biol 1980;85:899.) (Krogstad D, Schlesinger P. Acid-vesicle function, intracellular pathogens, and the action of chloroquine against Plasmodium Falciparum. NEJM 1987;317(9):542-49.) suggests that this is not unlikely.

HCQ is also known to inhibit platelet aggregation, (Winocour P, Kinlough-Rathbone R, Mustard J. The effect of the phospholipase inhibitor mepacrine on platelet aggregation, the platelet release reaction and fibrinogen binding to the platelet surface. Thrombos. Haemostas 1981;45(3):257-62.) and is commonly used as prophylaxis against thromboembolism after total hip replacement at some centers. See, Loudon J. Hydroxychloroquine and postoperative thromboembolism after total hip replacement. American Journal of Medicine 1988;85(4A):57-61. These actions take on significance given the recent report of increased platelet activation as an accompaniment of nocturnal asthma and the suggestion that platelets (or platelet-derived factors) may play a significant role in the genesis of asthma. See, Goldie R, Pedersen K. Mechanisms of increased airway microvascular permeability: role in airway inflammation and obstruction. Clin Exp Pharmaocol Physiol 1995;22:387-96. See, Gresele P, Dottorini M, Selli M, et al. Altered platelet function associated with the bronchial hyperresponsiveness accompanying nocturnal asthma. JACI 1993;91(4):894-902.

Viral infections have been implicated in the genesis of asthmatic exacerbations. See, Busse W, Swenson C, Borden E, et al. The effect of influenza A virus on leukocyte histamine release. Journal of Allergy and Clinical Immunology 1983;71:382-88. See, Chonmaitree T, Lett-Brown M, Grant J. Respiratory viruses induce production of histamine-releasing factor by mononuclear leukocytes: a possible role in the mechanism of virus-induced asthma. Journal of Infectious Diseases 1991;164:592-94. See, Hogg J. Persistent and latent viral infections in the pathology of asthma. American Review of Respiratory Disease 1992;145(2(supplement, part 2 of 2)):S7-9. The recent finding by Dr. Sperber and co-workers that HCQ inhibits infection by influenza and adenovirus (personal communication) and that HCQ actively inhibits replication of HIV virus suggests that the anti-viral properties of this drug may be relevant in this regard. See, Sperber K, Stecher V, Mayer L. Hydroxychloroquine inhibits HIV-1 replication in T cells and monocytes. FASEB J 1990:A2265. See, Sperber K, Kalb T, Stecher V, et al. Inhibition of HIV-1 replication by hydroxychloroquine in T cells and monocytes. AIDS Research and Human Retroviruses 1993;9(1):91-8. See, Tsai W-P, Nara P, Kung H-F, et al. Inhibition of human immunodeficiency virus infectivity by chloroquine. Aids Research and Human Retroviruses 1990;6(4):481-93.

In addition, variety of immuno-modulating effects on cell mediated immunity have also been noted including decreased lymphocyte proliferative responses, (Hurvitz D, Hirschhorn K. Suppression of “in vitro” lymphocyte responses by chloroquine. NEJM 1965;273:23.) decreased natural killer cell action, (Ausiello C, Barbieri P, Spagnoli G, et al. In vivo effects of chloroquine treatment on spontaneous and interferon-induced natural killer activities in rheumatoid arthritis patients. Clin Exp Rheum 1986;1:255.) and inhibition of granulomatous inflammation. See, DeSimone D, Brilliant H, Basile J, et al. Granulomatous infiltration of the talus and abnormal vitamin D and calcium metabolism in a patient with sarcoidosis: successful treatment with hydroxychloroquine. Am J Med 1989;87:694-96. These findings suggest a possible wider application for HCQ including such diseases as sarcoidosis, especially in view of recent reports of increased secretion of IL-1 and IL-6 by alveolar macrophages in patients with this disease. See, Steffen M, Petersen J, Oldigs M, et al. Increased secretion of tumor necrosis factor-alpha, interleukin-1-beta and interleukin-6 by alveolar macrophages from patients with sarcoidosis. JACI 1993;91(4):939-49.

In terms of safety, as compared with other anti-inflammatory and immunosuppressive medications currently under investigation for the treatment of asthma (as cited above), oral HCQ is safer and better tolerated according to two recent meta-analyses. See, Felson D, Anderson J, Meenan R. Comparing second line drugs for RA: Efficacy/toxicity tradeoffs. Arthritis and Rheumatism 1991;34(9S):S53(abstract #122). See, Fries J, Williams C, Bloch D. The relative toxicity of disease modifying anti-rheumatic drugs (DMARDS). Arthritis and Rheumatism 1991;34(9S):S54 (abstract #128).

As mentioned above, Hydroxychloroquine is currently available only by oral dosing. It is used as primary therapy as a disease-modifying agent anti-rheumatic agent for such diseases as rheumatoid arthritis and lupus erythematosus where it has achieved recognition for a low side effect profile and cost. See, Ostensen M. Treatment with immunosuppressive and disease modifying drugs during pregnancy and lactation. Am J Reprod Immunol 1992;28:148-52. See, Parke A. Antimalarial drugs, pregnancy and lactation. Lupus 1993;2:S21-3. In comparison with currently available alternative therapies, its safety profile recommends its use in women exposed to pregnancy during treatment and in some more severe cases even during gestation. Recent reports suggest that HCQ exerts its effects in a way distinct from other anti-inflammatory agents since HCQ may resolve some disease manifestations not affected by drugs such as methotrexate. See, Combe B, Didry C, Gutierrez M, et al. Accelerated nodulosis and systemic manifestations during methotrexate therapy for rheumatoid arthritis. Eur J Med 1993; 2 (153-6).

In addition, small studies and anecdotal report suggest a use for HCQ in the following disease entities: scleritis and uveitis associated with inflammatory bowel disease; sarcoidosis; atopic dermatitis; morphea; Q fever; and the hypergammaglobulinemia of Waldenstrom associated with systemic lupus erythematosus (Soukasian S, Foster C, Raizman M. Treatment strategies for scleritis and uveitis associated with inflammatory bowel disease. Am J Opthalmol 1994;118:601-11.) (Mathur A, Kremer J. Immunopathology, rheumatic features, and the therapy of sarcoidosis. Curr Opin Rheumatol 1992;4:76-80.) (Smith K. Hydroxychloroquine is useful in the management of atopic dermatitis. Br J Dermatol 1992;126:93-4.) (Peterson L, Nelson A, Su W. Classification of morphea. Mayo Clin Proc 1995;70:1068-76.) (Queyrel V, Bosseray A, Leclercq P, et al. Q fever and anutoimmunity. Ann Med Interne Paris 1995;146:132-3.) (Habib G, Stimmer M, Quismorio F. Hypergammaglobinemia of Waldenstrom associated with systemic lupus erythematosus. Lupus 1995;4:19-22.).

### SUMMARY OF THE INVENTION

This invention represents and is directed to the treatment of various disease states and/or an improved delivery of HCQ. The invention overcomes certain well-known problems and deficiencies in current therapies and treatment regimes.

Accordingly, it is an object of the present invention to provide for either oral or topical application of HCQ as an agent in the treatment of a variety of inflammatory pulmonary diseases, including asthma, as well as allergic and various skin disorders.

It can also be an object of this invention to provide a topical drug and/or therapy not dependent upon metabolic transformation of an active drug.

It can also be an object of this invention to provide for the treatment of such diseases, including those mentioned above, by way of a gradual onset of action consistent with a known active concentration of HCQ in the lung over time.

It can also be an object of the present invention to provide a variety of therapeutic advantages through use of HCQ as part of a topical application.

It can also be an object of the present invention to facilitate a more immediate concentration of HCQ, as compared to the oral deliveries of the prior art, such that the onset of therapeutic action is hastened.

It can also be an object of the present invention to provide for topical application of HCQ such that, in tissues where oral administration of HCQ does not result in appreciable concentrations thereof, local tissue concentrations may reach therapeutic levels.

It is an also an object of the present invention to further reduce the toxicity associated with HCQ, by decreasing the total dosage needed to achieve therapeutic response.

It is also an object of the present invention to provide for a formulation of HCQ to facilitate topical delivery and treatment of a variety of disease states including, but not limited to, allergic bronchopulmonary aspergillosis, allergic rhinitis, episclerigis, atopic dermatitis, inflammatory biodisease, sarcoidosis, allergic conjunctivitis, and scleritis.

It will be understood by those skilled in the art that one or more aspects of this invention can meet certain objectives, while one or more other aspects can meet certain other objectives. Each objective may not apply equally, in all instances, to every aspect of the present invention. In this regard, the aforementioned objects—as well as those aspects and features which follow—can be viewed in the alternative with respect to any one aspect of the present invention.

Other objects, features and advantages of the present invention will be apparent from this Summary and will be readily apparent to those skilled in the art having a knowledge of HCQ and various structurally-and therapeutically-related compounds including, other anti-malarials. Such objects, features, benefits and advantages will be apparent from the above as taken in conjunction with the accompanying summary, data and all reasonable inferences to be drawn therefrom.

Perennial bronchial asthma is a common disease pathologically marked by hyper-reactive airways, inflammation and edema of the bronchial epithelium and mucus secretory abnormalities. The clinical spectrum of asthma is quite broad—ranging from patients who have only very mild evanescent attacks to patients who have severe unremitting and often disabling asthma only poorly responsive to treatment with intense multi-drug therapy.

For patients with severe asthma, there are many problems with current therapy. First, patients must follow complicated medical schedules employing as many as four different metered-dose inhalers. Second, since the therapeutic half-life of most of these drugs is relatively short, the diurnal variation in disease severity frequently causes nocturnal attacks in many patients. Oral theophylline, which does have the benefit of a longer half-life, causes frequent gastrointestinal side-effects and has a relatively narrow therapeutic window, which necessitates careful monitoring to plasma levels to avoid more serious toxicity. Lastly, from a theoretical view, the majority of the drugs used in the treatment of asthma, including theophylline, beta-2 atagonists, and anticholinergics, are only spasmolytic agents which do not alter patterns of bronchial hyperactivity and have little or no effect on the inflammatory aspect of asthma. The limitations in the current therapeutic armamentarium are emphasized by the fact that over the last ten to fifteen years, both the hospitalization and mortality rates have actually risen, despite the introduction of several new anti-asthmatic drugs.

Recent asthma research has focused on the central role on airway inflammation in the pathogenesis of severe chronic asthma. See, Kay A B. Asthma and Inflammation. Journal of Allergy and Clinical Immunology 1991;87(5):893-910. See, Djukanovic R, Roche W R, Wilson J W, et al. Mucosal inflammation in asthma. American Review of Respiratory Disease 1990;142:434-457. See, Bousquet J, Chanez P, Lacoste J Y, et al. Eosinophilic inflammation in asthma. New England Journal of Medicine 1990;323(15):1033-39. These changes include increased mucus secretion, bronchial epithelial denudation and edema, basement membrane thickening and infiltration with eosinophils and other inflammatory cells as well as submucosal collagen deposition. As a consequence, mucociliary transport is depressed and mucus plugging of airways may ensue. The chemical mediators that underlie these reactions have been elucidated and are now known to include not only histamine and chemotactic factors, but also mast cell products such as the leukotrienes and PAF. It is these latter mediators that appear to be most closely associated with the “late phase” reactions characteristic of severe chronic asthma. See, Barnes P. New concepts in the pathogenesis of bronchial hyperresponsiveness and asthma. JACI 1989;83(6):1013-1026. See, Cockcroft D. Airway Hyperresponsiveness and Late Asthmatic Responses. Chest 1988;94(July 1):178-180.

However, of the drugs currently indicated for the treatment of asthma, only cromolyn sodium, nedocromil sodium and corticosteroids have specific anti-inflammatory actions. Unfortunately, while cromolyn and nedocromil have the capability to prevent late-phase reactions, their actions appear to be relatively mild and they are useful only in mild cases of asthma or as prophylaxis for challenge-induced bronchospasm (e.g., exercise). See, Cockcroft D W. Therapy for airway inflammation in asthma. Journal of Allergy and Clinical Immunology 1991;87(5):914-919.

Thus the corticosteroid family of drugs has unique actions in the treatment of asthma: active reduction of bronchial edema and mucus secretion, with demonstrable reduction of bronchial hyperactivity. See, Fox R I, Chan E, Benton L, et al. Treatment of Primary Sjogren's Syndrome with Hydroxychloroquine. A J Med 1988;85(suppl 4A):62-7. Furthermore, corticosteroids appear to synergize with spasmolytic agents in promoting relaxation of bronchial smooth muscle spasm. These actions have made corticosteroids, both in the inhaled topically active forms and in the systemic oral and parenteral forms, the cornerstone drug in the treatment of chronic or acute asthma. Unfortunately, the inhaled topically-active corticosteroids currently available in the United States appear to have limited efficacy and symptoms are not sufficiently controlled in many patients. Systemic corticosteroid treatment is potentially more effective, but is complicated by multiple and severe toxicities.

For this reason, several recent clinical trials have investigated the role of immuno-modulating, anti-inflammatory treatments in the treatment of asthma. See, Bernstein D I, Bernstein I L, Bodenheimer S S, et al. An Open Study of Auranofin in the Treatment of Steroid-Dependent Asthma. JACI 1988;81(1):6-15. See, Dyer P D, Vaughan T R, Weber R W. Methotrexate in the treatment of steroid-dependent asthma. Journal of Allergy and Clinical Immunology 1991;88(2):208-212. See, Mazer B D, Gelfand E W. An open-label study of high-dose intravenous immunoglobulin in severe childhood asthma. Journal of Allergy and Clinical Immunology 1991;87(5):976-983. 71. See, Mullarkey M F, Blumenstein B A, Andrade W P, et al. Methotrexate in the Treatment of Corticosteroid-Dependent Asthma. NEJM 1988;318(10):603-607. See, Mullarkey M F, Webb D R, Pardee N, E. Methotrexate in the Treatment of Steroid-Dependent Asthma. Ann Allergy 1986;56(April):347-350. See, Szefler S J, Rose J Q, Ellis E F, et al. The effect of troleandomycin on methylprednisolone elimination. Journal of Allergy and Clinical Immunology 1988;66:447-51. See, Shiner R J, Nunn A J, Chung K F, et al. Randomized, double-blind, placebo-controlled trial of methotrexate in steroid-dependent asthma. Lancet 1990;336:137-140. See, Klaustermeyer W B, Noritake D T, Kwong F K. Chrysotherapy in the Treatment of Corticosteroid-dependent Asthma. JACI 1987;79(5):720-25. See, Zeiger R S, Schatz M, Sperling W, et al. Efficacy of troleandomycin in outpatients with severe, corticosteroid-dependent asthma. Journal of Allergy and Clinical Immunology 1980;66:438-46. Some of these drugs, particularly methotrexate, have demonstrated or likely ‘steroid-sparing’ effects in the treatment of corticosteroid-dependent asthma. Unfortunately, these agents have toxic side effects potentially as serious as those of corticosteroids and this has limited their desirability as therapeutic agents. Other treatments, such as intravenous immune globulin (IVIG) or plasmapheresis are an economically unfeasible alternative.

The effects of the immuno-modulating agent, HCQ, on both steroid-dependent and severe symptomatic non-steroid-dependent asthmatics have been observed in open trials. See, Sherman B, Charous B. Short-term effects of hydroxychloroquine (HCQ) on IgE levels and obstructive airways disease. Journal of Allergy and Clinical Immunology 1992;89(1:2):286 (abstract); See, Charous B. An open study of hydroxychloroquine in the treatment of severe symptomatic or corticosteroid-dependent asthma. Annals of Allergy 1990;65(1):53; See, Charous B. Effectiveness of long-term treatment of severe asthma with hydroxychloroquine (HCQ). Annals New York Academy of Science 1991;629:432(abstract); and Charous B. Long-term treatment of severe asthma with hydroxychloroquine (HCQ). Annals of Allergy 1992;68(1):80 (abstract #40). The initial trial involved four severe symptomatic non-steroid dependent and seven steroid-dependent asthmatics in an open label 28-week trial at doses of 300-400 mg/d. Evaluation measures included daily symptom diary scores of six symptoms, subjective global evaluation, spirometric flow studies, and cumulative monthly steroid dosages. For the group as whole, mean FVC and FEV-1 increased 10% and 17% respectively (p<0.01) and the mean of each of six symptom scores declined significantly (p<0.05). Among the non-steroid-dependent asthmatics, the mean FVC and FEV-1 rose 19% and 32% respectively (p<0.05) and mean symptom scores for four of five symptoms declined from baseline (p<0.05). Among the steroid-dependent patients, cumulative mean monthly steroid dosage required decreased 50% from 383 mg at entry to 191 mg at week 28 (p<0.01), mean FVC and FEV-1 increased by 7% and 9%, although neither of these changes achieved significance, and two of five symptom scores improved (p<0.05). These improvements could not be explained by increased compliance to medication schedules since diaries indicated a fall in frequency of inhaler usage. In addition, no significant effect was noted on plasma theophylline levels. Comparison of pretreatment IgE levels to levels during treatment in ten patients demonstrated a fall from a mean of 645 to 339 IU/ml (p<0.05).

Follow-up observations carried out over the last four years among patients treated with HCQ continue to encourage further research. In Table I and II, data concerning the course of 23 steroid-dependent asthmatics (SDA) who have been followed for at least six months on therapy is presented. Spirometric data from these patients is summarized on page 2 of Table I. In the summary, two data groups are reported: the first includes all data points generated and compares each time point with t=0; the second is restricted to the 17 patients for whom data is complete at all time points. The findings are similar for both groups: both FVC and FEV-1 increase both a significant amount (about 10%) and to a degree that is statistically significant as well (using Wilcoxon Signed-Rank probability testing). These increases occurred despite a taper in prednisone outlined in Table I and detailed in Table II. Average daily oral steroid dosage was reduced by 60%, while objective spirometric flow measures and general health improved markedly.

Changes in severity of asthma in non-steroid-dependent asthmatics (NSDA) are depicted in Table III. In Table III, spirometric flow data is presented. Again, very significant improvements in forced vital capacity and FEV-1 are noted among the 11 patients studied. Probability testing (Wilcoxon Signed-Rank) in this group achieves significant change from baseline measures by month six and continued improvement is noted throughout the time of treatment.

Due to the uncontrolled nature of these observations, the possibility of placebo effects cannot be excluded. However, several considerations strongly argue that the improvements seen are real:

1] All of these were patients selected for a trial of HCQ due to their failure to respond to multi-drug regimens in the past, and introduction of a variety of alternate agents such as Atrovent or changes in inhaled steroid doses failed to elicit any long-acting placebo effect.

2] Few noticeable effects were seen during the first three months of therapy and significant effects appeared only after three months in the vast majority of these severe asthmatics. In neither the NSDA group (Table I) nor in the SDA group (Table II) did spirometric flows (as measured by FEV-1) improve to a statistically significant degree at three months. This delayed onset of action would be most unusual for a placebo effect, but on the other hand is quite consistent with the known onset of action of HCQ in other diseases, such as rheumatoid arthritis. In Table IV, data regarding the frequency of rescue-intervention in the NSDA group is reviewed. Again, when normalized rates are compared, (mean column 1 versus mean column 5 and 6), no significant fall in rescue-interventions occurs until after three months therapy. The course of patient KV, presented in Table V is illustrative of the reproducibility of the delay in onset of action seen in severe asthmatics. This patient was started on HCQ due to very labile asthma requiring frequent ER visits and oral steroid bursts (see Table VI). On HCQ she did better, but became pregnant (against medical advice) and the drug was discontinued. Subsequently, she did quite poorly necessitating restart of alternate-day oral steroids which were maintained during the latter part of her pregnancy. HCQ was restarted at her request following delivery. Again, no clear effect in KV was noted after three months, but after seven months substantial improvement was noted despite appreciable reduction in the dose of her alternate-daily oral steroids.

3] The improvements seen with initiation of HCQ therapy do not appear to wane even after more than two years of treatment; placebo effects rarely are this long-lived. Furthermore, several patients who have had relapses as their HCQ doses were discontinued or reduced appear to respond to resumption of full doses.

4] Improvement in patient performance and symptoms cannot be attributed to increased compliance with other asthma medications. This point was carefully reviewed in the initial study which demonstrated an actual fall in the use of concurrent medications while on HCQ. Other patients enrolled since the original study report a fall in the need for concurrent medications, and at least two patients (including the one serious relapse already noted) have felt well enough to virtually stop using what are clearly necessary adjunctive medications. Almost all patients have been able to convert to more acceptable BID or TID dosing regimens formerly not tolerated.

5] Symptomatic improvement, which is certainly subjective and thus most responsive to placebo effect, has been so dramatically altered as to suggest the improvement is real. Involuntary symptoms, such as nocturnal awakening, which were long-standing problems in many of these patients for years prior to HCQ, become a rare event. The need for “rescue” doses of steroids has fallen impressively even as patients have become much more active.

6] The objective improvements in spirometric studies corroborate these other less formal measurement indices. Most investigations of the therapeutic effects of new agents involve asthmatic patients who are not under any treatment with any anti-inflammatory medication. The fact that addition of HCQ yielded such improvements in a group of patients ostensibly enrolled in a “maximal” therapeutic program makes these changes even more impressive.

7] The degree of reduction in steroid dependence noted among SDA patients is quite similar to that reported in a variety of both double-blinded and uncontrolled studies investigating the utility of other anti-inflammatory medications such as Cyclosporine, Methotrexate, Dapsone, IVIG and oral gold salts.

Observations concerning HCQ have also included pre- and post-treatment IgE levels, summarized in Table VI. In general treatment with HCQ led to substantial reduction in serum IgE levels, amounting to approximately a 40% decrease. In patients with normal IgE levels, changes were not significant either in amount (19%) nor by statistical criteria. Significant declines in IgE were virtually completely confined to that segment of the patients who entered treatment with elevated IgE levels. Thus HCQ appears to act as an immuno-modulator for circulating IgE rather than simply as an immuno-suppressive. These clinical observations are supported by in vitro studies carried out recently by Seggev and co-workers. See, Seggev J, Hudig D, Hamilton R. Inhibition of IL-4 driven IgE synthesis by peripheral blood lymphocytes (PBL) in vitro with hydroxchloroquine (HCQ). J Immunol 1993;150(8):62A.

These findings may have important implications in the consideration of possible topical utility of this agent in the treatment of allergic diseases, including asthma. First, atopy, expressed as an elevated IgE, appears to be highly associated with the development of asthma. See, Tollerud D, O'Connor G, Sparrow D, et al. Asthma, hay fever and phlegm production associated with distinct patterns of allergy skin test reactivity, eosinophilia, and serum IgE levels. Am Rev Respir Dis 1991;144:776-81. See, Burrows B, Martinez F, Halonen M, et al. Association of asthma with serum IgE levels and skin-test reactivity to allergens. NEJM 1989;320(5):271-77. In particular, sensitivity to house dust mite allergen (Der. p I) is a predictor of the subsequent development of asthma in children. See, Sporik R, Holgate S, Platts-Mills T, et al. Exposure to house-dust mite allergen (Der p I) and the development of asthma in childhood. NEJM 1990;323(8):502-07. Thus the immuno-modulating effect of a topically administered agent which reduces IgE production, such as HCQ, may be useful to help reduce the stimulus to bronchial inflammation caused by allergic reactions to these and other inhalants.

Second, evidence increasingly indicates that specific IgE which causes allergic rhinitis may be locally produced. See, Huggins K, Brostoff J. Local production of specific IgE antibodies in allergic-rhinitis patients with perennial rhinitis. Lancet 1975;2:148-50. See, Platts-Mills T. Local production of IgG, IgA, IgE antibodies in grass pollen hay fever. J Immunol 1979;12:2218-25. See, Takenaka H, Kusumi T, Mizukoshi O. In vitro synthesis of IgE antibody by human tosil mononuclear cells. Acta Otolaryngo 1988;454:133-137. See, Merrett T, Houri M, Mayer A, et al. Measurement of specific IgE antibodies in nasal secretion. Evidence for local production. Clin Allergy 1976;6:69-73. See, Liu C-M, Shun C-T, Song H-C, et al. Antigen-specific IgE antibody and cytogram in mucosa of the nose and sinus. Am J Rhinology 1993;7(3):111-115. Topical agents which can reduce the stimulus to produce IgE by either blocking antigen processing (a known action of HCQ) or by blocking its production would appear to have a potentially valuable role in the treatment of allergic rhinitis or conjunctivitis.

While data presented are derived from uncontrolled observations, they are quite suggestive of a strong anti-asthmatic effect by HCQ. Multiple reasons have been forwarded which argue against these observations as only representing placebo effect, these include most importantly: 1] the delayed onset of action typical of HCQ but uncommon for placebo; 2] the lack of prior placebo effect in this group of patients; 3] objective data of change; and 4] changes similar in degree and kind to initiation of treatment with other systemic anti-inflammatory agents. Many of the mechanisms of action known for HCQ appear to be relevant to this disease process.

The widespread nature of its anti-inflammatory effects on mediators and cells thought relevant to pathogenesis of asthma and allergic disease, coupled with evidence of a gradual onset of action consistent with known active concentration of drug in the lung over time suggests that topical application of this agent may prove efficacious for a variety of inflammatory pulmonary diseases including asthma and for allergic and skin disorders.

Further support for topical application and the treatment of various pulmonary disease states is available from the results of a double blind trial of HCQ in moderate symptomatic asthma.

Methods:

Symptomatic asthmatics (mean duration>11 years) on stable doses of at least 6 puffs of inhaled steroid per day and p.r.n. bronchodilator (BD) were studied. Following a 6 week baseline run-in, they were randomized to 30 weeks of HCQ (n=8) or placebo (n=9). Changes from baseline mean FEV-1 comprised the primary efficacy variable. Other objective variables included daily peak flows, BD usage and setum IgE level; symptom scores and physician global (safety and tolerance) assessments were also obtained. Subjective efficacy variables (rated from 0-3) include breathing restrictions (wheezing, coughing, chest tightness and/or breathlessness), revived need for BD at 6 hours, and nocturnal asthma.

Results:

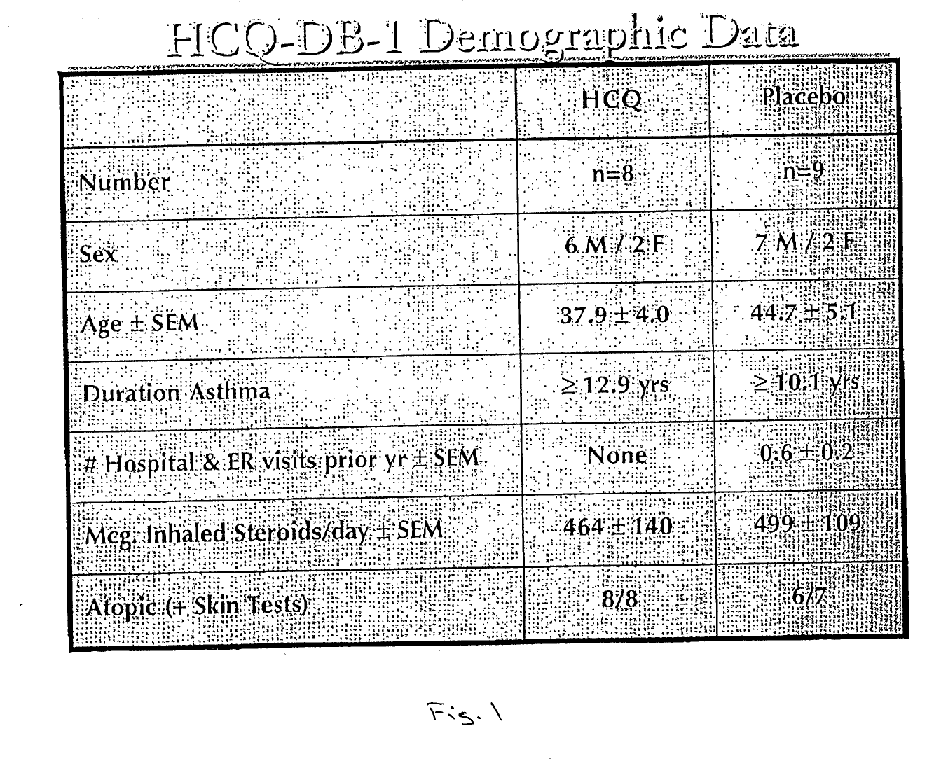

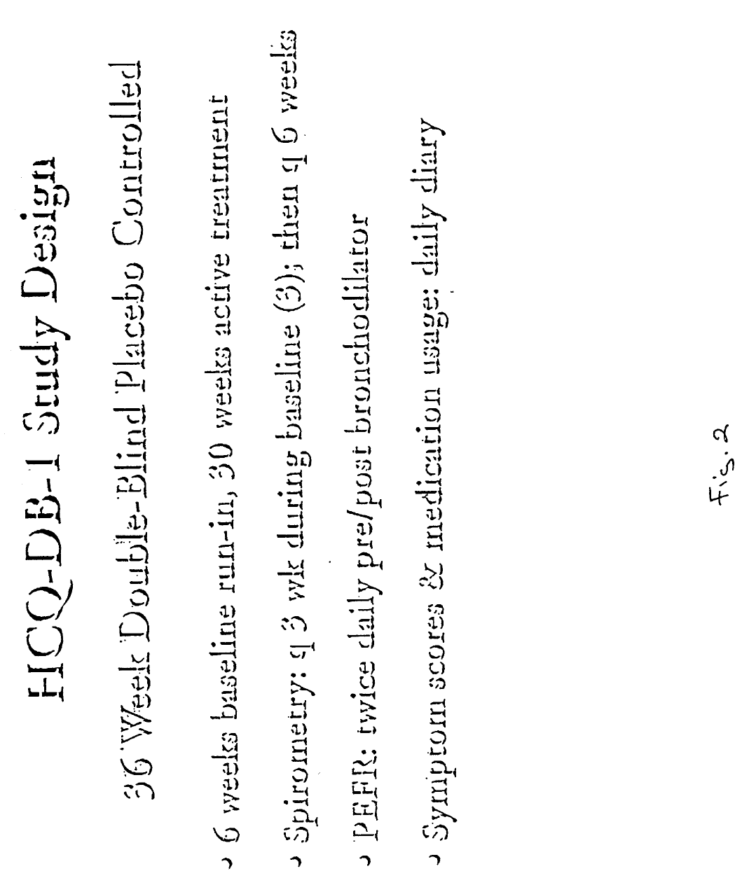

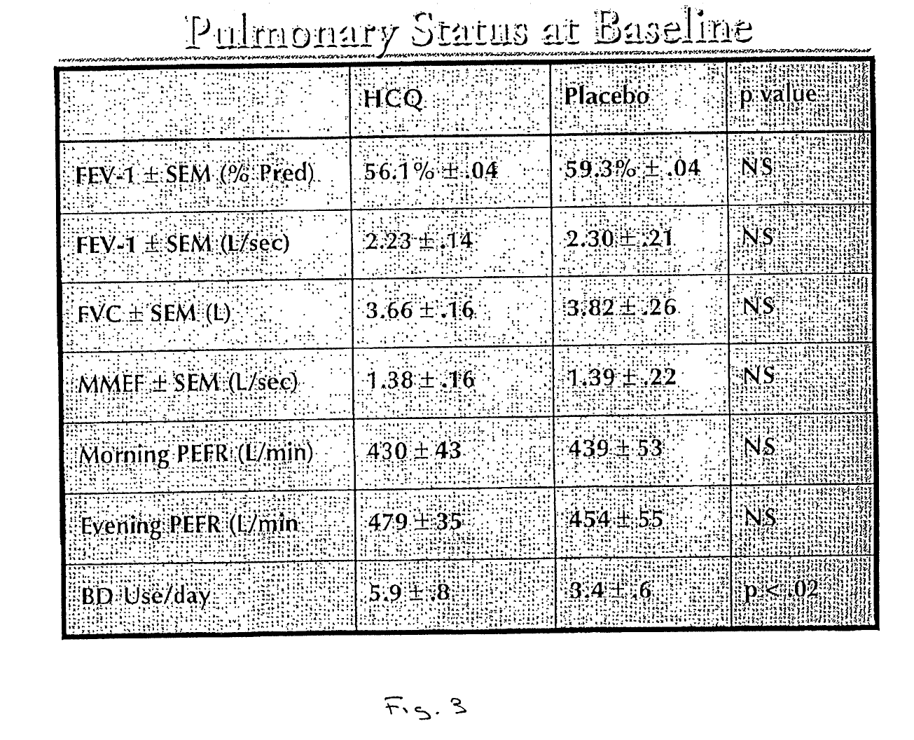

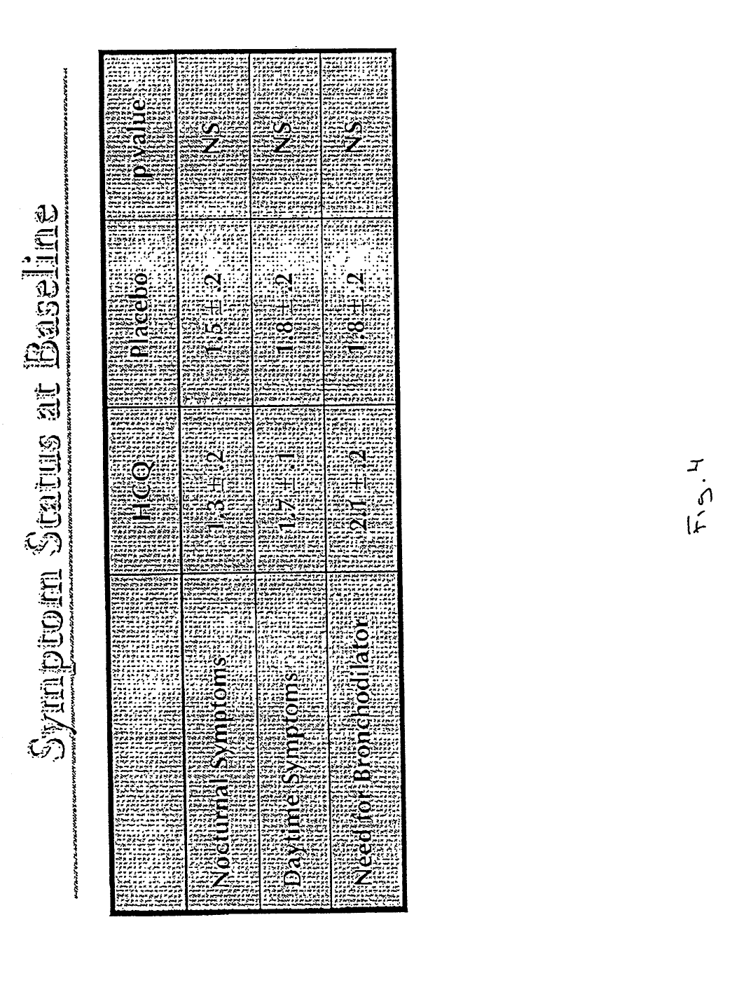

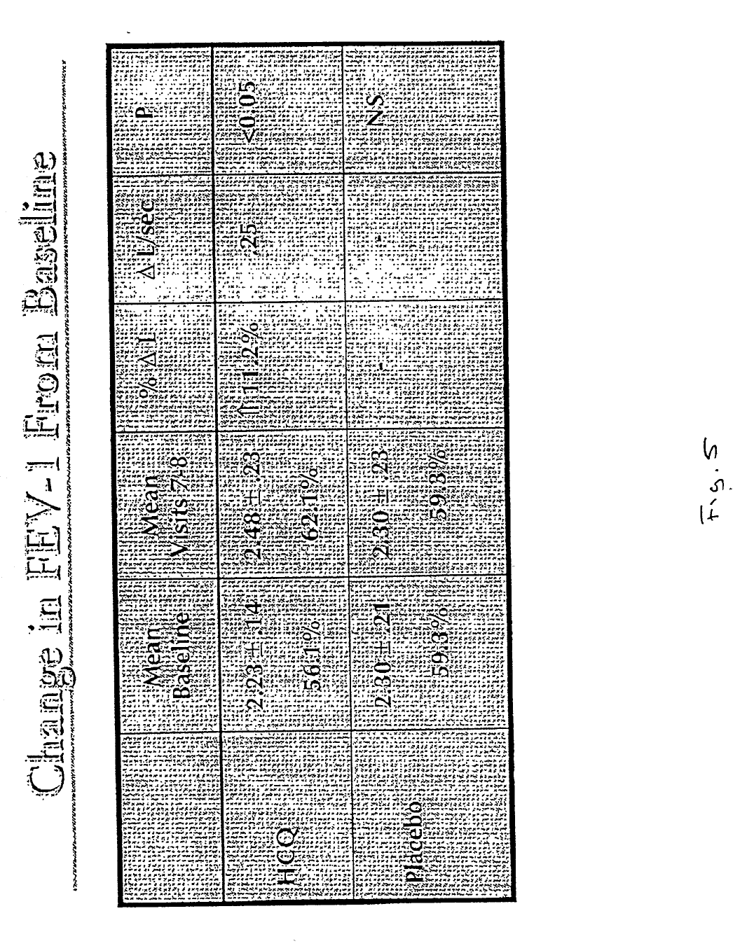

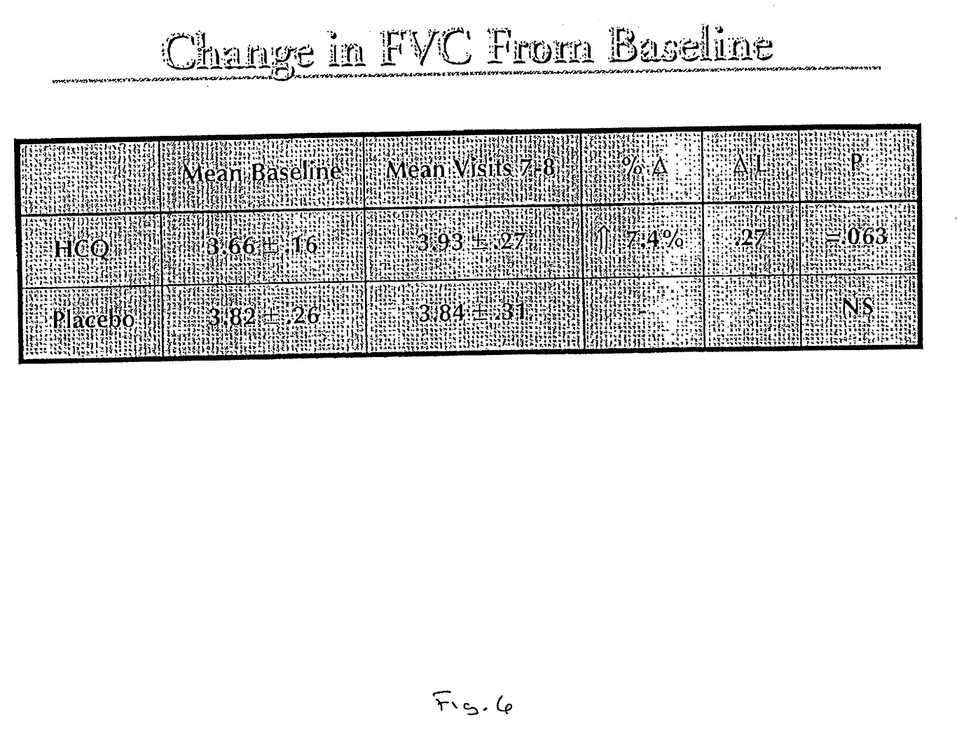

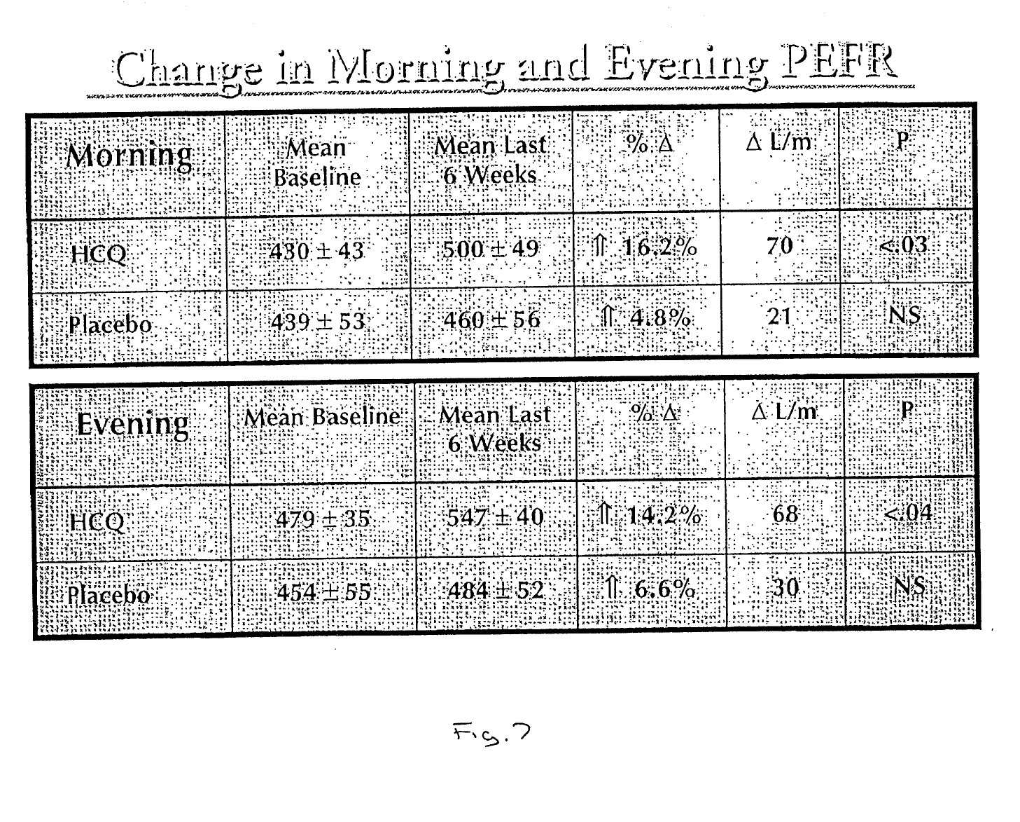

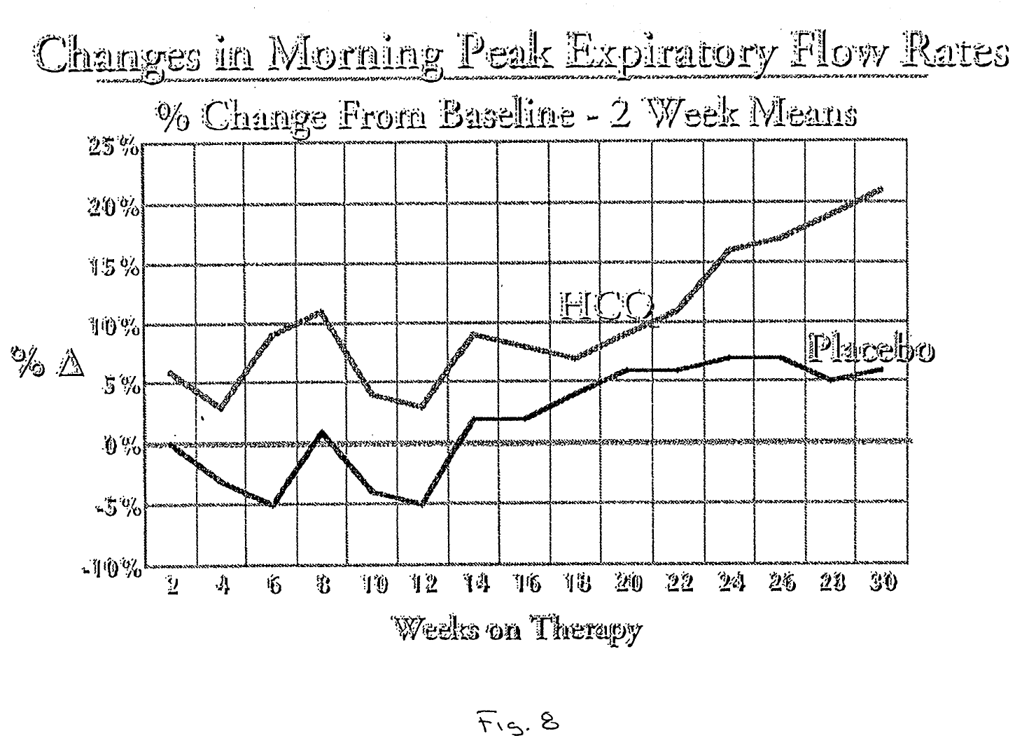

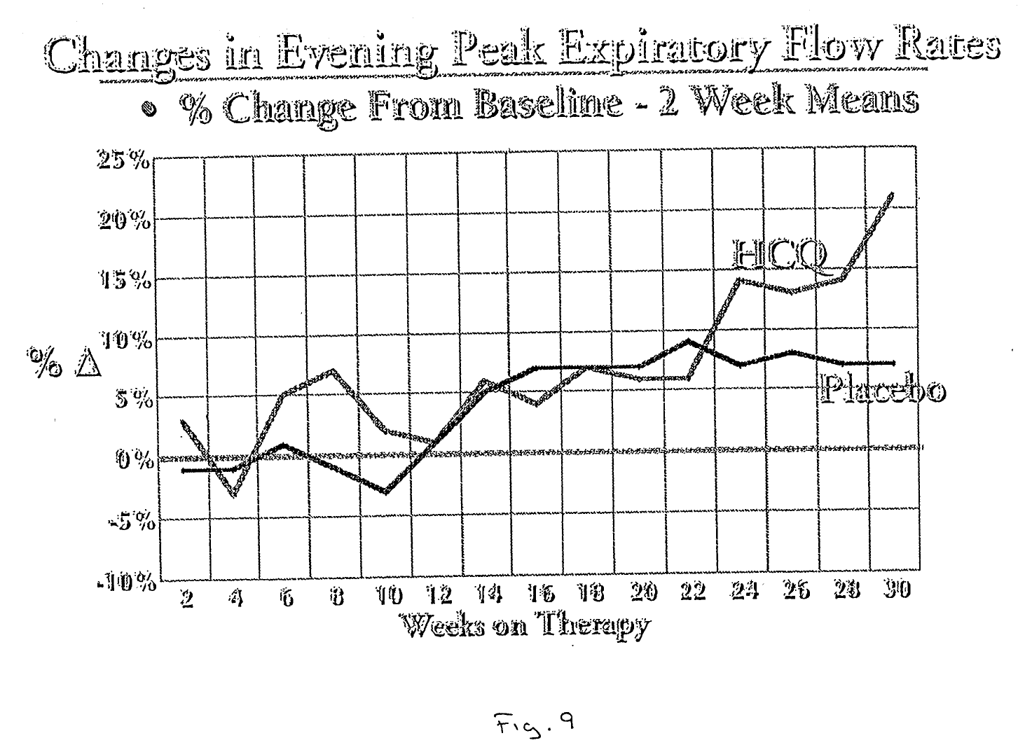

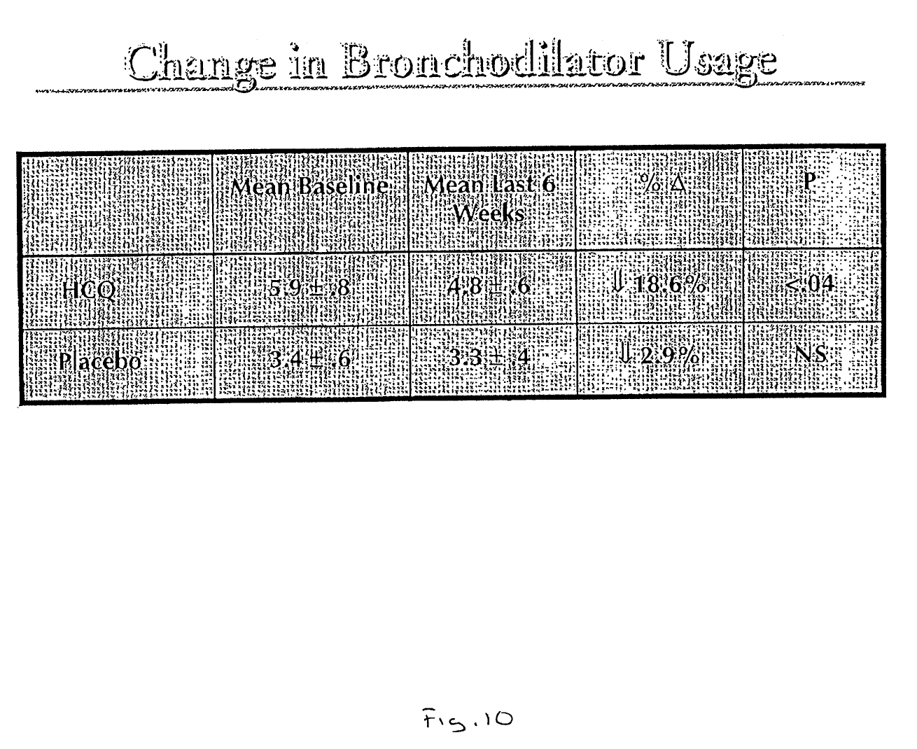

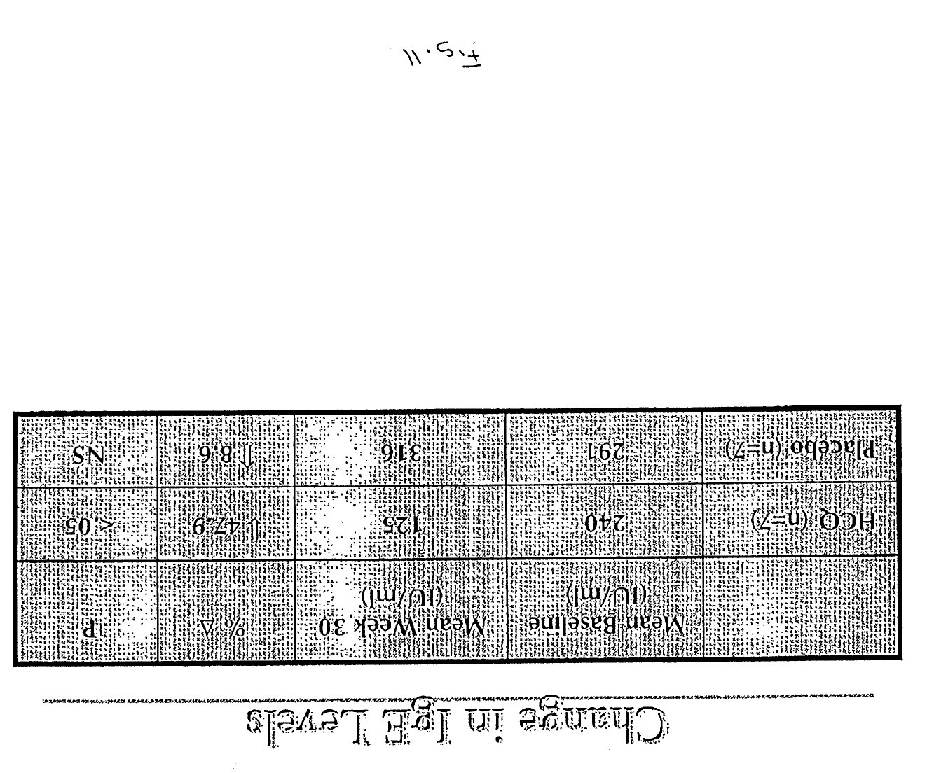

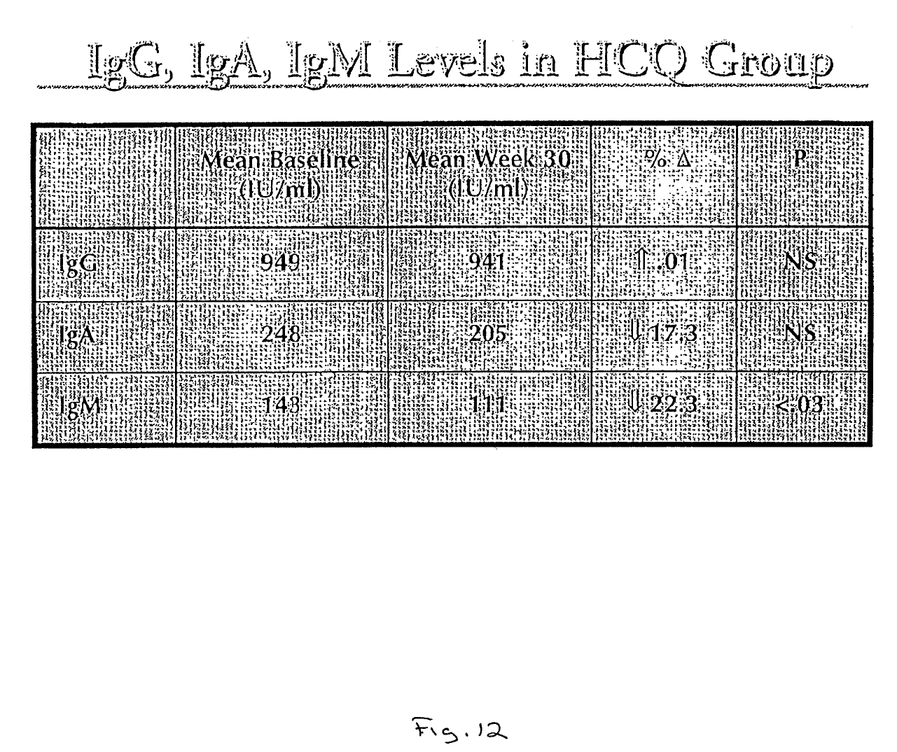

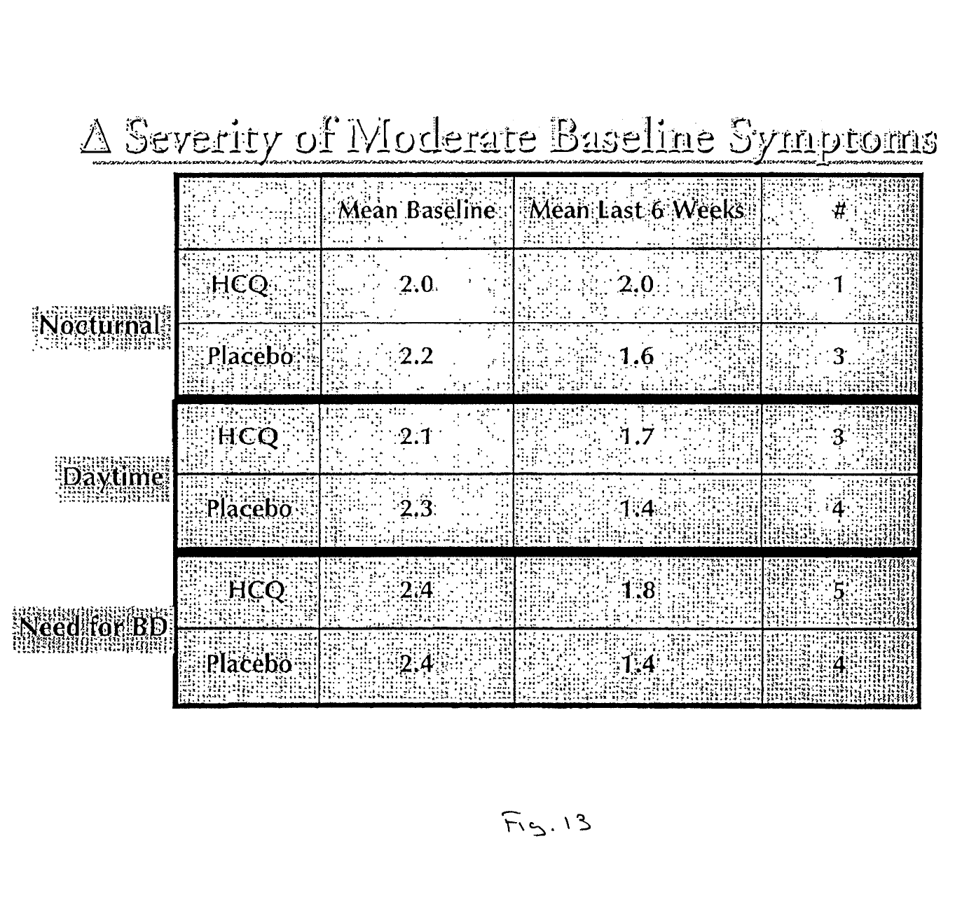

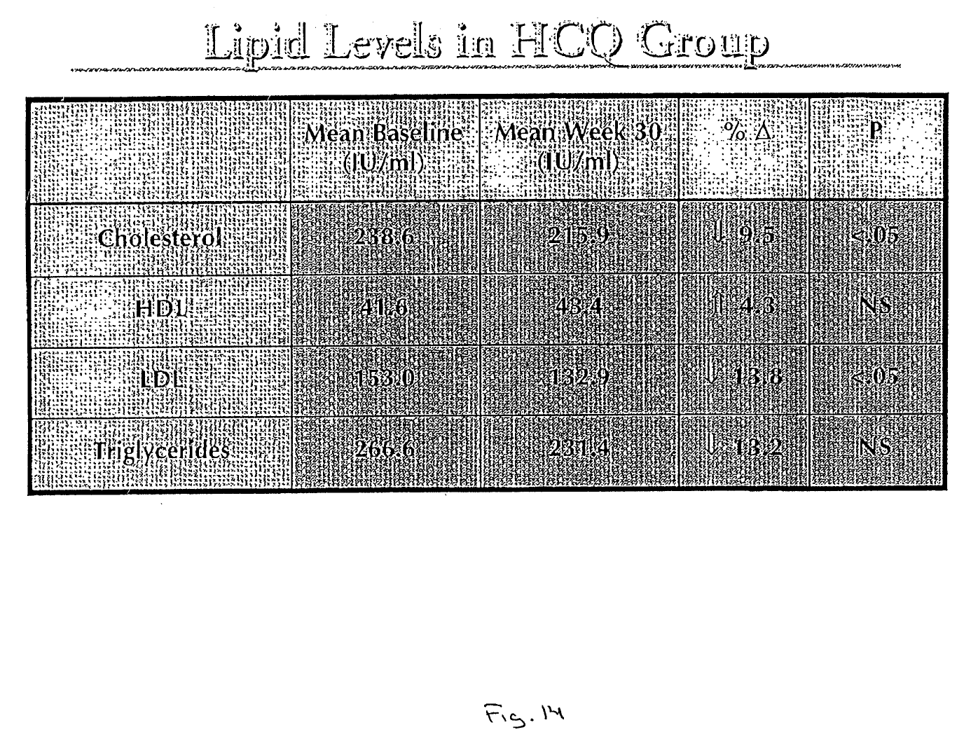

With reference to FIGS. 1-14, in the HCQ group, mean FEV-1 at the last 2 visits on therapy increased by 10.8% (p<0.05). Morning peak flows rose by 16.2% (p<0.03), evening peak flows by 14.2% (p<0.04) and BD usage fell by 18.6% (p<0.03). Mean IgE level declined 48% from 240 to 125 lU/ml (p<0.05). No significant change in any of these parameters occurred in the placebo group. Oral steroid rescue interventions were required in 4 patients on placebo and 2 on HCQ. HCQ was well tolerated without notable side-effects.

The results summarized in FIGS. 1-14 illustrate that HCQ provides a consistently producible delayed onset of action. Waning of effect is absent even after 24 months. Involuntary sytems, i.e., nocturnal asthma, are reduced. The steroid-sparing is similar to alternate experimental modalities. Prior trials of new drugs have failed to induce similar effect. These improvements are evidenced despite decreased usage of conventional prescriptions.

Conclusion:

Although the small sample population precludes definitive conclusions, these findings support our previous open label observations. The late improvement in the HCQ group is consistent with its known slow onset of action. Confirmatory studies are clearly warranted to investigate the anti-asthmatic, anti-allergic and possible disease-modifying effects of HCQ.

On balance, an anti-asthmatic effect is illustrated by incrased airway flows (PEFR & FEV-1) and decreased bronchodilator usage; immunomodulating effect on IgE levels; absence of side effects and drug interactions; mild hypocholesterolemic effects, sparing of HDL levels; and slow onset (peaking of presumed effect unknown).

Compared to alternative systemic agents, use of HCQ, as shown herein, provides unmatched safety, side effect; and cost profile; anti-asthmatic effects are matched only by CsA; and IgE immunomodulating effects are matched only by Au.

While the principles of this invention have been described in connection with specific embodiments, it should be understood clearly that these descriptions, along with the chosen tables and data provided therein, are made only by way of example and are not intended to limit the scope of this invention, in any manner. Other advantages and features of this invention will become apparent from an understanding of the invention, with the scope thereof determined by its reasonable equivalents, as would be understood by those skilled in the art.

In the claims:

1. A method of using a hydroxychloroquine composition in the treatment of inflammatory pulmonary disease states, said method comprising:
preparing a therapeutic dosage of said hydroxychloroqune composition; and
administering said dosage topically.

2. A method of using a hydroxychloroquine compound to reduce allergic antibody production, said method comprising:
preparing a therapeutic dosage of said hydroxychloroquine compound; and
administering said dosage topically.
